(19) United States
(12) Patent Application Publication
(10) Pub. No.: US 2023/0411023 A1
(43) Pub. Date: Dec. 21, 2023

(54) MACHINE LEARNING IDENTIFICATION OF AND COMMUNICATION TO MEDICAL PROFESSIONALS

(71) Applicant: PharmaForceIQ LLC, Hingham, MA (US)

(72) Inventor: Olatubosun Ogundero, Downingtown, PA (US)

(21) Appl. No.: 17/845,537

(22) Filed: Jun. 21, 2022

(51) Int. Cl. G16H 80/00; G16H 70/40; G16H 70/20; G06K 9/62

(52) U.S. Cl. CPC G16H 80/00; G16H 70/40; G16H 70/20; G06K 9/6256

(57) ABSTRACT

Introduced herein is a platform for determining machine learning communication/educating strategy. For example, the communication and educating strategy applies to introducing medical professionals to new, specialized medications. Medical professionals are categorized and/or characterized by their location, specialty, and therapy practice using claims data and labs data. Once a given medial professional is identified as one whom the specialized medication is useful to, an effectiveness model identifies a recommended channel and manner of educating the medical professional. The effectiveness model identifies one or more business metrics to be driven by a communication plan; generating one or more response functions of the business metrics by performing a machine learning process on a marketing dataset; and optimizing a spending subject of the marking plan subject to constraints to generate a marketing strategy based on multiple decision variables.

### TECHNICAL FIELD

The disclosure relates to machine learning and constrained optimization. More particularly, this disclosure relates to the use of machine learning to identify medical practitioners with target patients and a manner of engaging with those medical practitioners.

### BACKGROUND

Doctors prescribe pharmaceuticals based on those that they are aware of. To cause a doctor to prescribe something new to their patients, that doctor needs to be educated as to the new pharmaceuticals. Identifying which doctors to educate, when, and how is usually a nebulous affair. Presently these questions don't have simple answers and are addressed by humans often using arbitrary or convoluted systems.

### BRIEF DESCRIPTION OF THE DRAWINGS

FIG. 1A is an illustration of a dashboard graphic user interface.

FIG. 1B is an illustration of an individual doctor dashboard graphic user interface.

FIG. 2 is an illustration of a graphic that breaks down the prescriptions for a given medication by categorizations of medical professionals who have prescribed the medication.

FIG. 3 is a flowchart illustrating the implementation of claims and lab data.

FIG. 4 is a block diagram of an education event record.

FIG. 5 illustrates modules of a machine learning marketing strategy system according to the disclosed technology.

FIG. 6 illustrates a sample process of determining a machine learning communication strategy.

FIG. 7 is a flow chart illustrating an effectiveness rating process.

FIG. 8 is a diagrammatic representation of a machine in the example form of a computer system within which a set of instructions, for causing the machine to perform any one or more of the methodologies or modules discussed herein, may be executed.

### DETAILED DESCRIPTION

Doctors prescribe medication to patients. For a doctor to prescribe a given medication, the doctor has to (1) be aware of the medication (2) have a patient that the medication applies to. In addition, doctors tend to be busy, and the relevant education involved onboarding a given doctor should be tailored to channels and content that is effective with that given doctor. Disclosed herein is a platform that conducts both the identification of the right doctors to educate about a given medication, and the identification of how best to educate that right doctor.

The platform makes use of machine learning models that are trained with both claims and labs data for the identification of relevant doctors and impression data for each doctor for identifying how to educate that doctor. In an illustrative example, a doctor orders a panel of tests on a patient which return some values that indicate that the doctor has a patient who is a good candidate for a particular medication. The platform identifies that doctor as an ideal case to educate on the particular medication. In response, the platform further provides a suggested channel for education communication and a manner that communication should take.

For example, education communications tend to include information in a number of categories (e.g., 5) such as the safety information for the medication, the efficacy for the medication, dosing instructions for the medication, what the medication tests for or what test values should indicate its use, and what sorts of therapy techniques the medication is used with. Some doctors respond more readily to some of these above elements over others, and thus education communications should lead with the elements that the doctor finds most relevant. Further, the manner in which the doctor receives communications includes whether the doctor prefers to have branded communications and whether said branding should appear in subject lines or body content.

Indications on a given doctor regarding the channel and manner to educate a doctor are determined from impression and engagement data. For example, historical engagement and impression rates for different communications. Impression and engagement rates are determined based on a number of factors. These factors or key performance indicators (KPI) vary based on the channel employed. Further, different KPI apply at various stages. First, an engagement metric identifies whether the doctor opened/looked at a given communication. A later metric identifies whether the doctor responded or communicated based on the given communication. A third metric identifies whether the doctor prescribed the medication after having received the given communication.

Claims data tends to lag real time in a period measure in months whereas labs data tends to lag real time in a period measured in days. The period between a diagnosis and a prescription is also measured in days. Thus, when identifying the best doctors for education communications, the labs data is the most relevant and weighted more heavily. The labs data indicates candidate doctors with immediate needs, whereas claims data indicates candidate doctors with on-going or persistent needs.

The reason for the time lag on the associated data is because pharmaceutical companies tend to obtain these data sets in bulk from suppliers (for many needs). Waiting for the data to achieve a sufficient size threshold takes time and causes the lag. Therefore, the lag does not exist for any technical reason but rather an economic reason. As a result, in the future this lag may disappear as economic incentives changes. Changes in the pace of data enables additional embodiments of the present invention by enabling greater reliance on the data. In some embodiments, a “trigger” approach is an alternative to the “bulk” data approach. Specifically, a “trigger program” has less lag and is based on identifying a physician that is treating an eligible patient and ensuring the data supplier sends that “trigger” to the subject platform.

A marketer (e.g., of pharmaceuticals), in a general sense, has a decision as to how to spend their time/efforts. These decisions occur at both the macro level at the drug level and the micro level at the individual doctor level. For example, how much does the marketer spend at a first high-traffic web site, versus another high-traffic website, versus TV; effectively which channel? At the micro level, the marketer determines tighter details such as which doctors to work with and how to construct messages to those doctors. Herein described is a system and method to receive marketing data and process that data in order to determine a quantifiable value to each of these choices, thereby enabling the marketer to make better decisions.

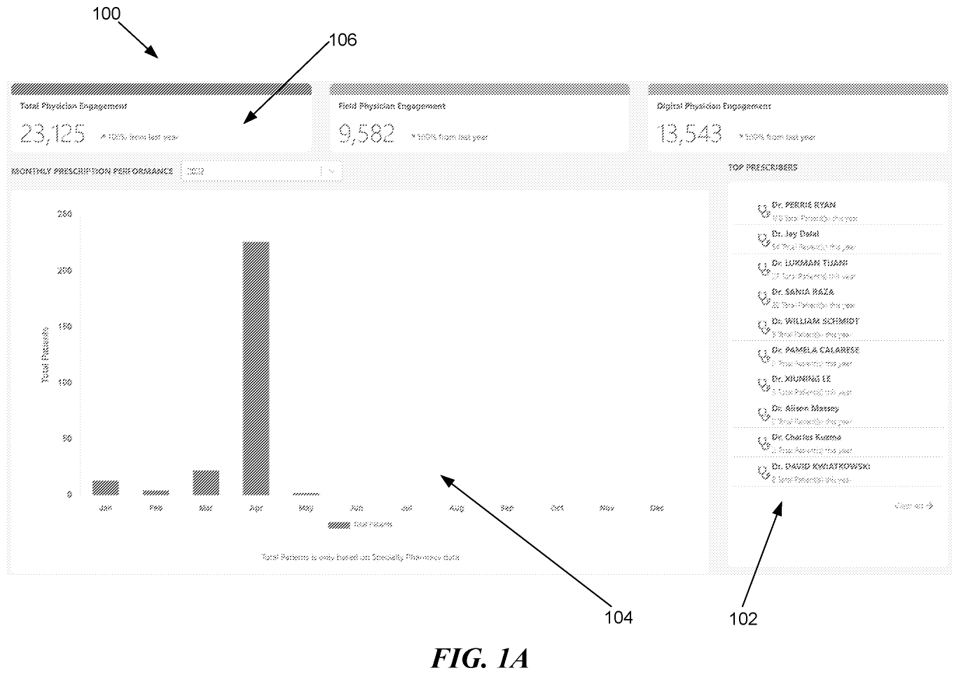

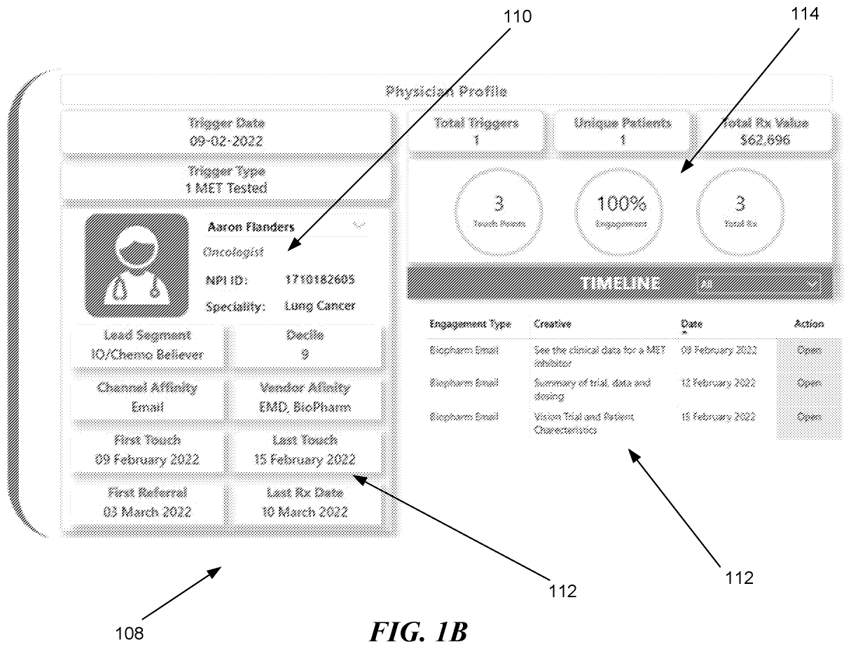

KPIs are discreet actions and are selected based on appropriateness to a given drug. Broad examples include: written prescriptions for the drug, value of a prescription of the drug, views/reads reports, website visits, incoming phone calls, or doctor responses (either favorable or unfavorable). Examples focused on interactions with websites or applications include: views, downloads, clicks, logins, or quantifiable functional activity (e.g., a user providing input to a). Chosen KPIs are measured as a function of time. The duration of the time may vary tremendously case-to-case. A given KPI data history may include only 2-3 weeks of data or 20 years of data. The system generates an effectiveness rating for each of the education communication events and/or granular components of the education event. This data may be plotted as depicted in FIG. 1.

### Graphical User Interface (GUI)

A user can log in to a graphical user interface to see a dashboard of metrics. For example, the dashboard can display to a user what the money they invested was spent on, and the costs for registering a targeted user on all of the indicated networks at different times. The information is generated automatically via the machine-learning module and optimization module and can be interpreted as the reasoning for an executable media decision.

FIG. 1A is an illustration of a dashboard graphic user interface 100. The dashboard 100 includes data regarding individual doctors 102 and the effect of education communication thereon. The dashboard includes graphs of various data elements such as patients prescribed a given medication over a period of time 104. The dashboard further illustrates engagement numbers across the platform 106. In some embodiments, the dashboard further includes targeted data regarding the amount of a given medication and/or the value of the prescriptions issued by a given medical professional.

FIG. 1B is an illustration of an individual doctor dashboard 108 graphic user interface. The individual doctor interface 108 is accessed by clicking on a doctor's name in the individual doctors 102 portion of the initial dashboard 100. The individual doctor dashboard 108 provides demographic information on the doctor 110, the history of communication and communication effectiveness history with the doctor 112, and the history of engagement with the subject medication 114. Using the individual doctor dashboard 108, a user is enabled to set alerts with respect to communication with the individual doctor.

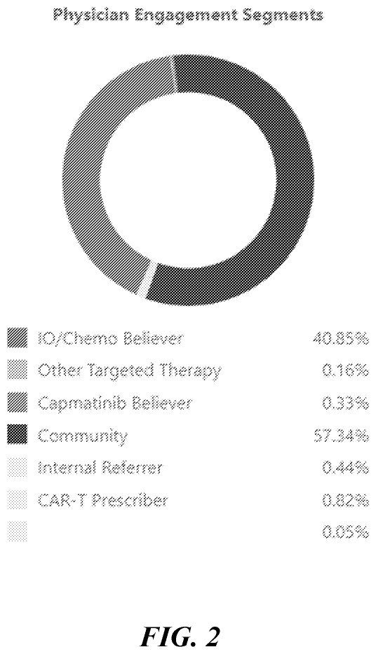

FIG. 2 is an illustration of a graphic that breaks down the prescriptions for a given medication by categorizations of medical professionals who have prescribed the medication. Specifically, the doctors are categorized by techniques or therapies that each conduct (as evidenced by claims data for each doctor). The claims data informs categories that each doctor falls into and subsequently how that doctor has operated with respect to a subject medication.

The dashboard further enables introduction of new look-alike doctors that are good candidates for education communications. A good candidate is one who is categorized into the same categories as those who have positive reactions to the subject medication. For example, the figure depicts that the subject medication is prescribed by many doctors who believe in subjecting their patients to chemotherapy. A new doctor, whom has had no interaction with the subject medication, but via claims data is shown to be a doctor of uses chemotherapy is a good candidate for education communications.

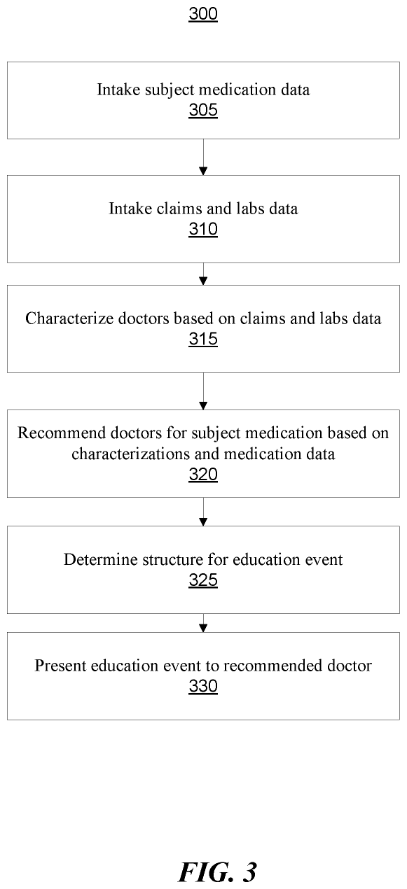

FIG. 3 is a flowchart illustrating the implementation of claims and lab data. In step 305, the platform receives data regarding a given medication. The data pertaining to the medication includes a set of therapies or procedures the medication is used in, similar therapies as to those the medication is used in, the specialties that the medication relates to, and the ideal circumstances (e.g., lab results) for a patient of the medication.

In step 310, the platform intakes a plurality of medical claims data and lab data. Claims data refers to the documents that indicate which procedures a doctor was performing and ordering for purposes of insurance claims. The data is anonymized with respect to the relevant patients but identifies the associated doctor. Claims data tends to lag real-time by a couple months, and thus does not inform the system in to present circumstances. Labs data refers to the results of tests ordered by the doctor for purposes of diagnosis. Labs data tends to lag real-time by one to three days. Accordingly, labs data is usable to recommend education events on the platform.

In step 315, the platform processes the intake of claims and lab data to classify or characterize the doctors associated therewith. As a result that labs data seeks to test for particular conditions, and procedures listed in claims data tend to relate to a given specialty, the platform identifies a specialty and an average patient for each doctor. In some embodiments, processing the intake of claims and lab data further includes a deduplication process that screens the intake data so as to not bias characterization of each doctor.

In step 320, the platform makes recommendations to educators of a subject medication are most relevant for that medication based on the data for that given medication and the characterization of each doctor. In some embodiments, the recommendation is based on the labs data (e.g., the doctor has a patient who is a prime candidate for prescription of the subject medication via labs results). In some embodiments, the recommendation is based on the claims data that indicate that the doctor frequently conducts therapies that the subject medication is used in. In some embodiments, the recommendation is based both on labs and claims data that indicate the doctor has patients that would use the subject medication.

The recommendation is based on output from a model, such as a trained machine leaning model (e.g., a hidden Markov model, a neural network, a convolutional neural network, or a few-shot model). In some embodiments, the recommendation is generated via heuristics designed according to the subject medication. The training for the model is performed using the claims and labs data in addition to doctor demographics.

In step 325, once a doctor has been recommended for an education event of the subject medication, the structure of the education event is determined by an effectiveness model (described further below). The structure of the event referring to the time, channel, and manner the education event is presented to the recommended doctor.

In step 330, the platform presents the education event to the recommended doctor.

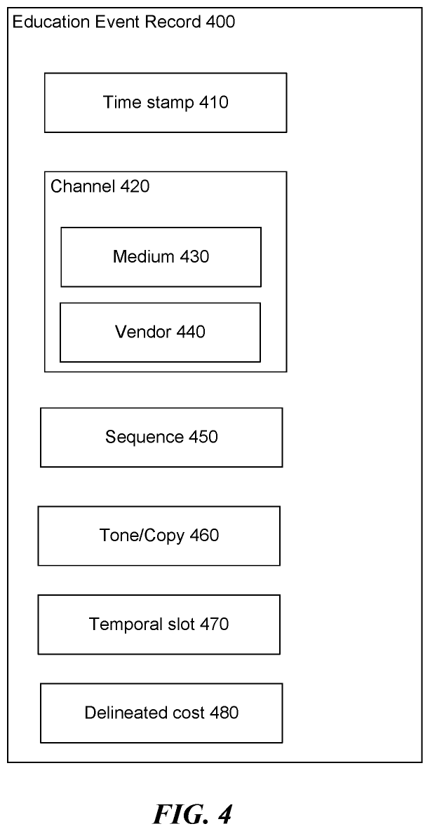

FIG. 4 is a block diagram of an education event record. Each education event includes a number of details to which an effectiveness is credited. In order to process necessary data, the system stores education event records 400 within a database. As previously discussed, an education event record for a given education event has a time stamp 410. The timestamp 410, like the rest of the data stored with a given education event record 400 is metadata.

Education events include a number of important metadata characteristics stored in an education event record 400. The channel 420 of the subject education event refers to the delivery method of the education event. The channel 420 further breaks down into the medium 430 used and the vendor 440 within the medium. Example mediums 420 include: email, text message, personal promotion (e.g., communication via a sales representative using virtual, phone call or in-person education), direct mail, physician led instruction (e.g., a key opinion leader such as physician who is very experienced in the field influences other physicians in their geography/network such as through seminars or continuing medical education), programmatic targeted web ads (e.g., targeting banner ads on physician's IP addresses), or website banners (e.g., targeted to all or a significant percentage of visitors to a particular website). The vendor 440 is the particular implementation of the selected medium 430. For example, a particular website, or a particular placement agency.

Another characteristic is the sequence 450 of the education event. Sequence 450 refers to the order of content within the education event. The content about a given medication is predetermined and largely fixed, however, the order in which that content is presented to a doctor effects the receipt thereof. Some doctors prefer education content to lead with particular details that they are more interested in. The tone 460 of the education event refers to whether a branding is applied along with the education event and if so, where to implement that branding.

The temporal slot 470 of the subject education event refers to when the education event occurs. Examples include, mornings, Fridays, seasonal, “halfway through” some subject work, or other suitable temporal placements known in the art. Embodiments of temporal placements slots 470 further refer to association with lab data. Specifically, some doctors are more receptive to education events in response to lab data that is relevant to the subject medication. Therefore, education events occurring within a threshold period of time of lab data is a portion of metadata stored to a given education event.

Finally, education includes an associated cost. Varying some of the above characteristics affects the cost. Thus, the cost is a delineated cost 480 that is attributable in part to each of the above characteristics. For example, a given vendor 440 may have a greater cost for certain mediums over others.

Each of the above characteristics of an education event record 400 provide a more granular look at each event. When placed along with KPI history data, an effectiveness value can be determined for these characteristics individually. This is performed by isolating particular characteristics. Where two events have similar characteristics with one variance, changes in the KPI history data are attributable to that difference in characteristic. The particular characteristic may be assigned the effectiveness of that difference in KPI.

Machine learning algorithms and hierarchical-based models are used to perform this signal processing step. The system parses through the KPI history data which is compared to the education event records 400 in order to test the characteristics in each education event record 400 and to optimize each.

In some cases, education events having identical or substantially similar characteristics generate different effectiveness results. In these circumstances the KPI data is used to improve the machine learning observation pool. The multiple effectiveness ratings may be averaged or used to generate a hierarchical model. The optimization process can be, e.g., a non-linear, combinatorial tradeoff optimization process over a large number (e.g., hundreds or thousands) of variables.

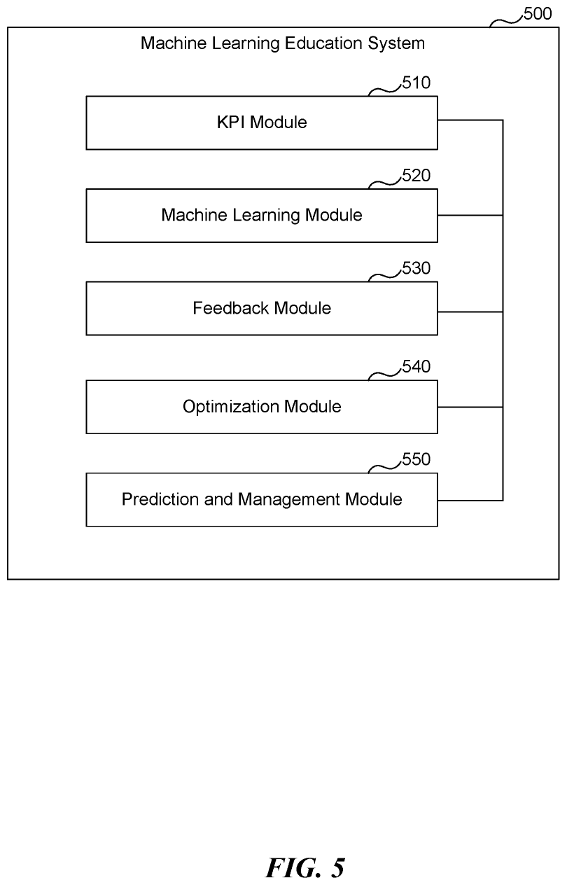

FIG. 5 illustrates modules of a machine learning marketing strategy system according to the disclosed technology. As illustrates in FIG. 5, the machine learning marketing system 500 includes a KPI module 510, a machine learning module 520, a feedback module 530, an optimization module 540, and a prediction and management module 550. The KPI module 510 defines the outcome KPIs (key performance indicators) that the marketing platforms (e.g., TV networks for serving advertisements) should drive. In some embodiments, the KPI module 510 does not necessarily focus dogmatically on a particular KPI, regardless of the advertiser or the circumstances. Instead, the KPI module 510 focuses on client KPIs that are measurable and are meaningful marketing outcomes. As a result, the marketing strategy system 500 can make education investment decisions based on the relationships of the KPIs to what is important, rather than just what is easy to measure.

The machine learning module 520 parses existing KPI history data to develop an effectiveness profile of given education events and/or more granular characteristics of education events. In embodiments where existing KPI history data is undeveloped, the machine learning module 520 makes use of external marketing data to complete an observation phase. The external marketing data chosen may relate to competing products/services or be purchasable set-top box (STB) data.

In a cold start situation, where a brand-new doctor who has never received an education event the system employs look-alike data. Doctors are characterized by their demographic data including claims data, lab data, and other demographic data. Where a new doctor to the system has existing claims and lab data, that doctor is compared to other existing platformed doctors who have similar claims and lab data. Where the doctor is new to practice, other data is still available to identify look-alikes. For example, other demographic data includes the doctor's specialty, the facility the doctors work at, the geographic region the doctor is in, and the licensing institution for that doctor.

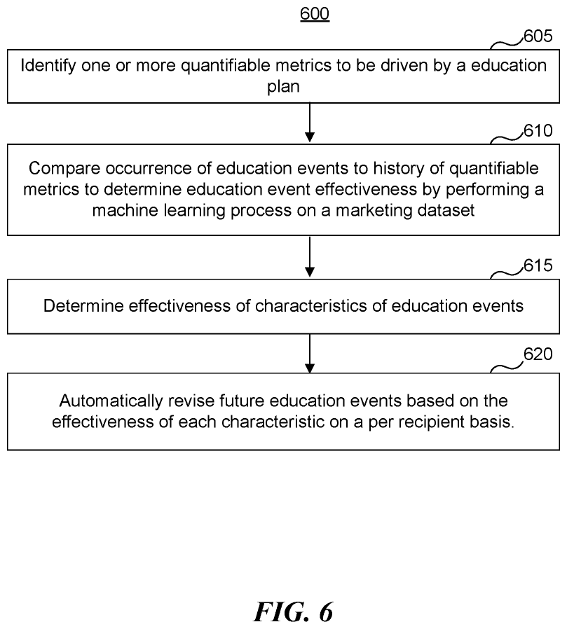

FIG. 6 illustrates a sample process of determining a machine-learning marketing strategy. FIG. 6 illustrates a sample process of analyzing marketing data using machine learning.

In step 605, the system identifies one or more quantifiable metrics from which effectiveness of education events is determined. This decision may be user generated or based on the product/service offered. In step 610, the system compares occurrence of education events to history of quantifiable metrics to determine education event effectiveness by performing a machine learning process on a marketing dataset.

Step 610 is a machine learning signal processing phase. Raw data is input into a machine learning module to be normalized in a signal processing stage. A parsing module can exist in the machine learning module to automatically parse through the data, both input and output data.

In step 615, based on the comparison of the quantifiable metric to individual education events, the system determines an effectiveness rating for each of the education events. Additionally, each education event is compared to other education events in order to isolate and determine effectiveness of characteristics of each education event.

In step 620, the system automatically revises future education events based on the effectiveness of each characteristic on a per recipient basis. For example, based on which education event characteristics were effective (as measured by the KPI), those characteristics are reused with future education events for that doctor and other look-alike doctors.

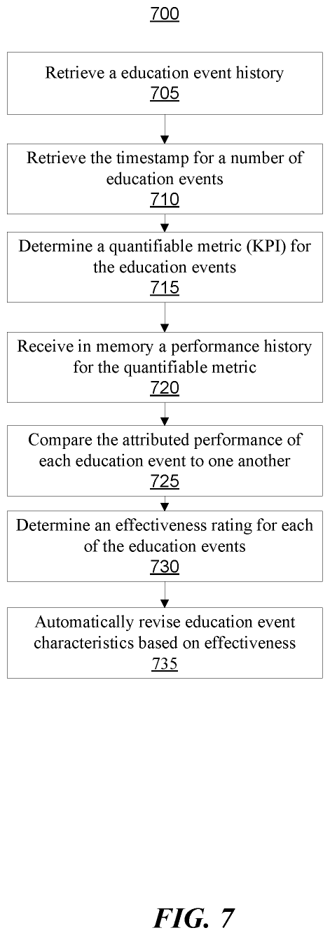

FIG. 7 is a flow chart illustrating an effectiveness rating process. In step 705, the system stores in memory an education event history. The education event history includes metadata of a plurality of education events. Among the metadata are details such as a timestamp, a medium, channel, venue, content, tone, magnitude, time slot, and cost.

In step 710, the system retrieves the timestamp for a number of education events. In step 715, the system determines a quantifiable metric (KPI) for the education events. In step 720, the system stores in memory a performance history for the quantifiable metric. The performance history includes the quantifiable metric measured as a function of time.

In step 725, the system assigns time periods to each education event based on changes in the performance history data occurring after the timestamp of the education events. In step 730, an effectiveness rating for each education event is determined based on the KPI connected to each respective education event. In step 735, the system automatically revises education events based on identifications of the most effective education events.

### Constrained Optimization

Within the optimization module, a user can prescribe constraints upon the media decisions, KPIs, and miscellaneous inputs. The constrained optimization module prompts the user to enter, into a graphic user interface, the values of importance. Based on the input values, the constrained optimization module performs a variation of the maximization process described previously. The constrained optimization module calculates the expected value of the function based on the constraints, then finds the parameter that maximizes the function such that it converges to a maximum likelihood estimate of a parameter. In other words, the constrained optimization will iterate through calculating the expected value and maximizing a parameter until it yields a best estimate.

### Exemplary Computer System

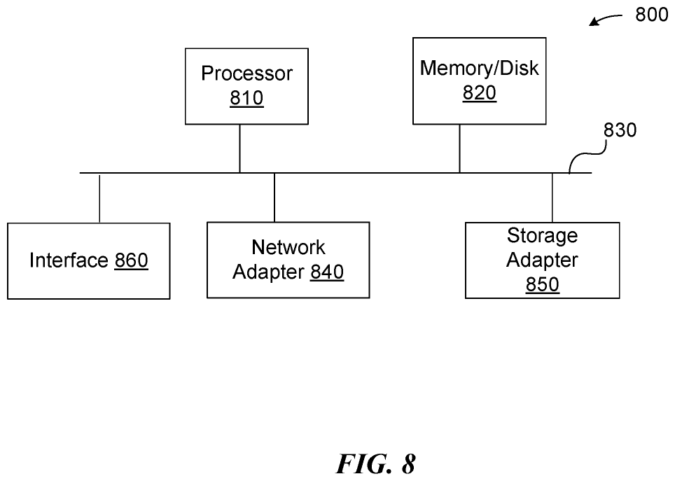

FIG. 8 is a diagrammatic representation of a machine in the example form of a computer system 800 within which a set of instructions, for causing the machine to perform any one or more of the methodologies or modules discussed herein, may be executed.

The computer system 800 includes a processor 810, memory and non-volatile memory 820, a communications BUS 830, a network adapter 840, a disk interface 850 and an interface device 860. Various common components (e.g., cache memory) are omitted for illustrative simplicity. The computer system 800 is intended to illustrate a hardware device on which any of the components described in the examples (and any other components described in this specification) can be implemented. The components of the computer system 800 are coupled together via the bus 830 or through some other known or convenient device.

This disclosure contemplates the computer system 800 taking any suitable physical form. As an example, and not by way of limitation, a computer system 800 may be an embedded computer system, a system-on-chip (SOC), a single-board computer system (SBC) (such as, for example, a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, or a combination of two or more of these. Where appropriate, computer system 800 may include one or more computer systems 800; be unitary or distributed; span multiple locations; span multiple machines; or reside in a cloud, which may include one or more cloud components in one or more networks. Where appropriate, one or more computer systems 800 may perform, without substantial spatial or temporal limitation, one or more steps of one or more methods described or illustrated herein. As an example, and not by way of limitation, one or more computer systems 800 may perform in real time or in batch mode one or more steps of one or more methods described or illustrated herein. One or more computer systems 800 may perform at different times or at different locations one or more steps of one or more methods, described or illustrated herein, where appropriate.

The processor may be, for example, a conventional microprocessor such as an Intel Pentium microprocessor or Motorola power PC microprocessor. One of skill in the relevant art will recognize that the terms “machine-readable (storage) medium” or “computer-readable (storage) medium” include any type of device that is accessible by the processor.

The memory is coupled to the processor by, for example, a bus. The memory can include, by way of example but not limitation, random access memory (RAM), such as dynamic RAM (DRAM) and static RAM (SRAM). The memory can be local, remote, or distributed.

The bus also couples the processor to the non-volatile memory and drive unit. The non-volatile memory is often a magnetic floppy or hard disk, a magnetic-optical disk, an optical disk, a read-only memory (ROM), such as a CD-ROM, EPROM, or EEPROM, a magnetic or optical card, or another form of storage for large amounts of data. Some of this data is often written, by a direct memory access process, into memory during execution of software in the computer 800. The non-volatile storage can be local, remote, or distributed. The non-volatile memory is optional because systems can be created with all applicable data available in memory. A typical computer system will usually include, at least, a processor, memory, and a device (e.g., a bus) coupling the memory to the processor.

The front-end server interfaces with a user interface, obtaining user inputs relating to, for example, desired KPIs. The front-end server can contain infrastructure to perform user management such as log-in information and advertising account information. In some embodiments, the front-end server can also contain a web server that communicates with a user interface. Some embodiments of the front-end server can contain a rendering module in which it can process a user-inputted request. It can load a page, a layout of the page with CSS and JavaScript, and content of the page.

A back-end server behaves as an intermediary between the front-end server, the database server, the machine learning module, and optimization module. The back-end server performs all the computations and processes input and sends this information back to the front-end server. In one embodiment, the back-end server can contain a data verification module that communicates with the database server to verify that the data stored in either the decision variables database or the KPI database is the most up-to-date data.

Software is typically stored in the non-volatile memory and/or the drive unit. Indeed, storing an entire large program in memory may not even be possible. Nevertheless, it should be understood that for software to run, if necessary, it is moved to a computer readable location appropriate for processing, and for illustrative purposes, that location is referred to as the memory. Even when software is moved to the memory for execution, the processor will typically make use of hardware registers to store values associated with the software, and local cache that, ideally, serves to speed up execution. As used herein, a software program is assumed to be stored at any known or convenient location (from non-volatile storage to hardware registers) when the software program is referred to as “implemented in a computer-readable medium.” A processor is considered to be “configured to execute a program” when at least one value associated with the program is stored in a register readable by the processor.

The bus also couples the processor to the network interface device. The interface can include one or more of a modem or network interface. It will be appreciated that a modem or network interface can be considered to be part of the computer system 800. The interface can include an analog modem, ISDN modem, cable modem, token ring interface, satellite transmission interface (e.g. “direct PC”), or other interfaces for coupling a computer system to other computer systems. The interface can include one or more input and/or output devices. The I/O devices can include, by way of example but not limitation, a keyboard, a mouse or other pointing device, disk drives, printers, a scanner, and other input and/or output devices, including a display device. The display device can include, by way of example but not limitation, a cathode ray tube (CRT), liquid crystal display (LCD), or some other applicable known or convenient display device. For simplicity, it is assumed that controllers of any devices not depicted in the example of FIG. 5 reside in the interface.

In operation, the computer system 800 can be controlled by operating system software that includes a file management system, such as a disk operating system. One example of operating system software with associated file management system software is the family of operating systems known as Windows® from Microsoft Corporation of Redmond, Washington, and their associated file management systems. Another example of operating system software with its associated file management system software is the Linux™ operating system, and its associated file management system. The file management system is typically stored in the non-volatile memory and/or drive unit and causes the processor to execute the various acts required by the operating system to input and output data and to store data in the memory, including storing files on the non-volatile memory and/or drive unit.

The algorithms and displays presented herein are not inherently related to any particular computer or other apparatus. Various general purpose systems may be used with programs in accordance with the teachings herein, or it may prove convenient to construct more specialized apparatus to perform the methods of some embodiments. The required structure for a variety of these systems will appear from the description below. In addition, the techniques are not described with reference to any particular programming language, and various embodiments may thus be implemented using a variety of programming languages.

In alternative embodiments, the machine operates as a stand-alone device or may be connected (e.g., networked) to other machines. In a networked deployment, the machine may operate in the capacity of a server or a client machine in a client-server network environment, or as a peer machine in a peer-to-peer (or distributed) network environment.

The machine may be a server computer, a client computer, a personal computer (PC), a tablet PC, a laptop computer, a set-top box (STB), a personal digital assistant (PDA), a cellular telephone, an iPhone, a Blackberry, a processor, a telephone, a web appliance, a network router, switch or bridge, or any machine capable of executing a set of instructions (sequential or otherwise) that specify actions to be taken by that machine.

While the machine-readable medium or machine-readable storage medium is shown in an exemplary embodiment to be a single medium, the term “machine-readable medium” and “machine-readable storage medium” should be taken to include a single medium or multiple media (e.g., a centralized or distributed database, and/or associated caches and servers) that store the one or more sets of instructions. The term “machine-readable medium” and “machine-readable storage medium” shall also be taken to include any medium that is capable of storing, encoding or carrying a set of instructions for execution by the machine, and that cause the machine to perform any one or more of the methodologies or modules of the presently disclosed technique and innovation.

In general, the routines executed to implement the embodiments of the disclosure may be implemented as part of an operating system or a specific application, component, program, object, module or sequence of instructions, referred to as “computer programs.” The computer programs typically comprise one or more instructions set at various times in various memory and storage devices in a computer, and that, when read and executed by one or more processing units or processors in a computer, cause the computer to perform operations to execute elements involving the various aspects of the disclosure.

Further examples of machine-readable storage media, machine-readable media, or computer-readable (storage) media include, but are not limited to, recordable-type media such as volatile and non-volatile memory devices, floppy and other removable disks, hard disk drives, optical disks (e.g., Compact Disk Read-Only Memory (CD ROMS), Digital Versatile Disks, (DVDs), etc.), among others, and transmission-type media such as digital and analog communication links.

In some circumstances, operation of a memory device, such as a change in state from a binary one to a binary zero or vice-versa, for example, may comprise a transformation, such as a physical transformation. With particular types of memory devices, such a physical transformation may comprise a physical transformation of an article to a different state or thing. For example, but without limitation, for some types of memory devices, a change in state may involve an accumulation and storage of charge or a release of stored charge. Likewise, in other memory devices, a change of state may comprise a physical change or transformation in magnetic orientation or a physical change or transformation in molecular structure, such as from crystalline to amorphous or vice versa. The foregoing is not intended to be an exhaustive list in which a change in state for a binary one to a binary zero or vice-versa in a memory device may comprise a transformation, such as a physical transformation; rather, the foregoing is intended as illustrative examples.

A storage medium typically may be non-transitory or comprise a non-transitory device. In this context, a non-transitory storage medium may include a device that is tangible, meaning that the device has a concrete physical form, although the device may change its physical state. Thus, for example, non-transitory refers to a device remaining tangible despite this change in state.

Reference in this specification to “one embodiment” or “an embodiment” means that a particular feature, structure, or characteristic described in connection with the embodiment is included in at least one embodiment of the disclosure. The appearances of the phrase “in one embodiment” in various places in the specification are not necessarily all referring to the same embodiment, nor are separate or alternative embodiments mutually exclusive of other embodiments. Moreover, various features are described that may be exhibited by some embodiments and not by others. Similarly, various requirements are described that may be requirements for some embodiments but not others.

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements. The coupling or connection between the elements can be physical, logical, or a combination thereof. For example, two devices may be coupled directly, or via one or more intermediary channels or devices. As another example, devices may be coupled in such a way that information can be passed there between, while not sharing any physical connection with one another. Additionally, the words “herein,” “above,” “below,” and words of similar import, when used in this application, shall refer to this application as a whole and not to any particular portions of this application. Where the context permits, words in the Detailed Description using the singular or plural number may also include the plural or singular number respectively. The word “or,” in reference to a list of two or more items, covers all of the following interpretations of the word: any of the items in the list, all of the items in the list, and any combination of the items in the list.

If the specification states a component or feature “may,” “can,” “could,” or “might” be included or have a characteristic, that particular component or feature is not required to be included or have the characteristic.

The term “module” refers broadly to software, hardware, or firmware components (or any combination thereof). Modules are typically functional components that can generate useful data or another output using specified input(s). A module may or may not be self-contained. An application program (also called an “application” or “app”) may include one or more modules, or a module may include one or more application programs.

The foregoing description of various embodiments of the claimed subject matter has been provided for the purposes of illustration and description. It is not intended to be exhaustive or to limit the claimed subject matter to the precise forms disclosed. Many modifications and variations will be apparent to one skilled in the art. Embodiments were chosen and described in order to best describe the principles of the invention and its practical applications, thereby enabling others skilled in the relevant art to understand the claimed subject matter, the various embodiments, and the various modifications that are suited to the particular uses contemplated.

While embodiments have been described in the context of fully-functioning computers and computer systems, those skilled in the art will appreciate that the various embodiments are capable of being distributed as a program product in a variety of forms, and that the disclosure applies equally regardless of the particular type of machine or computer-readable media used to actually effect the distribution.

Although the above Detailed Description describes certain embodiments and the best mode contemplated, no matter how detailed the above appears in text, the embodiments can be practiced in many ways. Details of the systems and methods may vary considerably in their implementation details, while still being encompassed by the specification. As noted above, particular terminology used when describing certain features or aspects of various embodiments should not be taken to imply that the terminology is being redefined herein to be restricted to any specific characteristics, features, or aspects of the invention with which that terminology is associated. In general, the terms used in the following claims should not be construed to limit the invention to the specific embodiments disclosed in the specification, unless those terms are explicitly defined herein. Accordingly, the actual scope of the invention encompasses not only the disclosed embodiments, but also all equivalent ways of practicing or implementing the embodiments under the claims.

The language used in the specification has been principally selected for readability and instructional purposes, and it may not have been selected to delineate or circumscribe the inventive subject matter. It is therefore intended that the scope of the invention be limited not by this Detailed Description, but rather by any claims that issue on an application based hereon. Accordingly, the disclosure of various embodiments is intended to be illustrative, but not limiting, of the scope of the embodiments, which is set forth in the following claims.

What is claimed is:

1. A computer-implemented method for determining measurable responses to communication events, comprising:
determining a quantifiable metric for a set of communication events, quantifiable metrics include any of:
views;
read receipts;
phone calls;
clicks;
prescriptions issued;
value of prescriptions issued; or
application interactions;
storing, in memory, a communication event history, the communication event history including metadata of a plurality of communication events, each communication event respectively including a content order, a channel, and a recipient;
normalizing the quantifiable metric across each channel;
training a model on a per recipient basis prioritizing the quantifiable metric based on variations on the content order and the channel as associated with each given recipient; and
generating a new communication event for a first recipient wherein the content order and the channel of the new communication event is based on a query to the model using the first recipient as input.

2. The method of claim 1, wherein the recipient of the communication event history includes demographic metadata, and the model does not include sufficient training data for the first recipient, the method further comprising:
generating look-a-like training data of the first recipient based on training data of other recipients that include matching demographic metadata.

3. The method of claim 2, wherein the demographic data includes a recipient location, a recipient employer, and a recipient field of work.

4. The method of claim 1, wherein the channel includes both a medium and a vendor associated with the medium.

5. The method of claim 4, wherein the medium of the channel includes any of:
email;
text message;
personal promotion;
direct mail;
physician led instruction;
programmatic targeted web ads; or
website banner.

6. The method of claim 1, further comprising:
generating a graphic user interface that depicts values of the quantifiable metric as attributed to the first recipient across the set of communication events.

7. The method of claim 1, wherein said training of the model further includes data indicating a relative cost of each potential channel and said generating the new communication is further based on the model evaluating the relative cost.

8. A computer-implemented system for determining measurable responses to communication events, comprising:
a memory configured to store a communication event history, the communication event history including metadata of a plurality of communication events, each communication event respectively including a content order, a channel, and a recipient;
a processor-implemented effectiveness model that is configured to determine a quantifiable metric for a set of communication events wherein the quantifiable metric is normalized across each channel, the effectiveness model trained on a per recipient basis prioritizing the quantifiable metric based on variations on the content order and the channel as associated with each given recipient, wherein quantifiable metrics include any of:
views;
read receipts;
phone calls;
clicks;
prescriptions issued;
value of prescriptions issued; or
application interactions; and
wherein the effectiveness model is further configured to generate a new communication event for a first recipient wherein the content order and the channel of the new communication event is based on a query to the effectiveness model using the first recipient as input.

9. The system of claim 8, wherein the recipient of the communication event history includes demographic metadata, and the model does not include sufficient training data for the first recipient, the effectiveness model further configured to:
generate look-a-like training data of the first recipient based on training data of other recipients that include matching demographic metadata.

10. The system of claim 9, wherein the demographic data includes a recipient location, a recipient employer, and a recipient field of work.

11. The system of claim 8, wherein the channel includes both a medium and a vendor associated with the medium.

12. The system of claim 11, wherein the medium of the channel includes any of:
email;
text message;
personal promotion;
direct mail;
physician led instruction;
programmatic targeted web ads; or
website banner.

13. The system of claim 8, wherein the effectiveness model is further configured to:
generate a graphic user interface that depicts values of the quantifiable metric as attributed to the first recipient across the set of communication events.

14. The system of claim 8, wherein said training of the model further includes data indicating a relative cost of each potential channel and said generating the new communication is further based on the model evaluating the relative cost.

15. A computer-implemented method for determining measurable responses to communication events, comprising:
storing, in memory, a communication event history, the communication event history including metadata of a plurality of communication events, each communication event respectively including a content order, a channel, and a recipient;
comparing a first communication event against a second communication event based on a quantifiable metric for a set of communication events, the first communication event and the second communication event each refer to a first recipient but have a different content order or different channel, wherein the quantifiable metric is normalized across each channel, quantifiable metrics include any of:
views;
read receipts;
phone calls;
clicks;
prescriptions issued;
value of prescriptions issued; or
application interactions;
training a model for the first recipient based on said comparing; and
generating a new communication event for the first recipient wherein the content order and the channel of the new communication event is based on a query to the model using the first recipient as input.

16. The method of claim 15, wherein the recipient of the communication event history includes demographic metadata, and the model does not include sufficient training data for the first recipient, the method further comprising:
generating look-a-like training data of the first recipient based on training data of other recipients that include matching demographic metadata.

17. The method of claim 16, wherein the demographic data includes a recipient location, a recipient employer, and a recipient field of work.

18. The method of claim 15, wherein the channel includes both a medium and a vendor associated with the medium.

19. The method of claim 18, wherein the medium of the channel includes any of:
email;
text message;
personal promotion;
direct mail;
physician led instruction;
programmatic targeted web ads; or
web site banner.

20. The method of claim 15, further comprising:
generating a graphic user interface that depicts values of the quantifiable metric as attributed to the first recipient across the set of communication events.
